(19) United States
(12) Patent Application Publication
(10) Pub. No.: US 2026/0325805 A1
(43) Pub. Date: Oct. 22, 2026

(54) FLEXIBLE SHUNT IMPLANTS

(71) Applicant: Edwards Lifesciences Corporation, Irvine, CA (US)

(72) Inventors: Daniel James Murray, Orange, CA (US); Eric Jason Noda, Rancho Santa Margarita, CA (US); Linda Thai, Mission Viejo, CA (US); Jonathan Andrew Lam, Anaheim Hills, CA (US)

(21) Appl. No.: 19/726,554

(22) Filed: Jun. 30, 2026

Related U.S. Application Data
(63) Continuation of application No. 18/336,265, filed on Jun. 16, 2023, now Pat. No. 12,691,266.
Application No. 18/336,265 is a continuation of application No. PCT/US2021/063773, filed on Dec. 16, 2021.
(60) Provisional application No. 63/199,323, filed on Dec. 18, 2020.

(51) Int. Cl. A61M 27/00; A61B 17/00; A61B 17/11; A61B 17/34

(52) U.S. Cl. CPC A61M 27/002; A61B 17/11; A61B 17/3415; A61B 2017/00252; A61B 2017/003; A61B 2017/00867; A61B 2017/1139

(57) ABSTRACT

A delivery system comprises a catheter configured to be delivered through a blood flow pathway of a heart and an implant configured to maintain an opening in a tissue wall to allow blood flow through the opening and into the blood flow pathway and be delivered via the catheter. The implant is at least partially composed of a flexible material to allow the implant to bend with the catheter.

### RELATED APPLICATION

This application is a continuation of U.S. patent application Ser. No. 18/336,265, filed Jun. 16, 2023, which is a continuation of International Patent Application No. PCT/US2021/063773, filed Dec. 16, 2021, which claims the benefit of U.S. Patent Application No. 63/199,323, filed Dec. 18, 2020, the entire disclosures of each of which are incorporated by reference for all purposes.

### BACKGROUND

### Field

The present invention relates generally to the field of medical devices and procedures.

### Description of Related Art

In percutaneous delivery systems for delivering certain medical implant devices to target locations at least in part through a patient's vasculature, certain anatomical and device dimensions can limit the size, shape, and/or configuration of medical implant devices delivered using such systems.

### SUMMARY

For purposes of summarizing the disclosure, certain aspects, advantages and novel features have been described herein. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

Some implementations of the present disclosure relate to a delivery system comprising a catheter configured to be delivered through a blood flow pathway of a heart and an implant configured to maintain an opening in a tissue wall to allow blood flow through the opening and into the blood flow pathway and be delivered via the catheter. The implant is at least partially composed of a flexible material to allow the implant to bend with the catheter.

The implant may be configured to be crimped around an outer surface of the catheter. In some embodiments, the implant is at least partially composed of braided cords. The implant may be at least partially composed of a coiled cord.

In some embodiments, the implant comprises a puncture element configured to puncture through a tissue wall to create the opening in the tissue wall. The puncture element may be further configured to anchor the implant to the tissue wall. In some embodiments, the implant further comprises an anchoring element and the puncture element and the anchoring element are configured to anchor to opposing sides of the tissue wall.

The implant may comprise a first tubular portion and a second tubular portion. In some embodiments, the first tubular portion is configured to bend away from the second tubular portion to allow the first tubular portion to enter the opening in the tissue wall. The second tubular portion may be configured to extend along the blood flow pathway and beyond the opening in the tissue wall.

In some embodiments, the first tubular portion and the second tubular portion are separate devices. The first tubular portion and the second tubular portion may extend from a common base portion. In some embodiments, the first tubular portion is at least partially composed of a shape-memory material.

The delivery system may further comprise a sheath configured to at least partially enclose the implant and retract to at least partially expose the implant and allow the implant to expand. In some embodiments, the blood flow pathway is a coronary sinus and the opening creates a flow pathway between a left atrium and the coronary sinus.

Some implementations of the present disclosure relate to a method comprising delivering an implant enclosed at least partially by a sheath to a coronary sinus via a right atrium. The implant is at least partially composed of braided or coiled cords to allow the implant to bend into the coronary sinus. The method further comprises puncturing a tissue wall of the coronary sinus to create an opening between a left atrium and the coronary sinus, retracting the sheath to expose the implant, and anchoring the implant at the opening. The implant is configured to maintain the opening.

The implant may comprise a puncture element. Puncturing the tissue wall may be performed using the puncture element.

In some embodiments, anchoring the implant at the opening involves contacting the tissue wall with the puncture element. The puncture element may be configured to establish a single point of contact with the tissue wall.

The implant may comprise a first tubular portion and a second tubular portion. Retracting the sheath may allow the first tubular portion to bend away from the second tubular portion.

In accordance with some implementations of the present disclosure, a medical implant comprises means for maintaining an opening through a tissue wall of a heart. The opening creates a blood flow path from a first chamber of a heart to a blood flow pathway of the heart. The means for maintaining the opening is/are configured to bend to facilitate delivery into the blood flow pathway and the first chamber. The medical implant further comprises means for anchoring to the tissue wall at the opening.

The medical implant may further comprise means for puncturing the tissue wall to create the opening.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements. However, it should be understood that the use of similar reference numbers in connection with multiple drawings does not necessarily imply similarity between respective embodiments associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some embodiments or configurations.

FIG. 1 illustrates several access pathways for maneuvering guidewires and/or catheters in and around the heart to deploy compressible implants in accordance with some embodiments.

FIG. 2 depicts a procedure for deploying implants in accordance with some embodiments.

FIGS. 3A and 3B illustrate a flexible shunt implant in accordance with some embodiments.

FIG. 4 (FIGS. 4-1 and 4-2) is a flow diagram illustrating a process for delivering and/or anchoring an implant to a treatment site in accordance with one or more embodiments of the present disclosure.

FIG. 5 (FIGS. 5-1 and 5-2) provides several images associated with the process of FIG. 4 to illustrate aspects of the process according to one or more implementations thereof.

FIG. 6 illustrates another example shunt implant in accordance with embodiments of the present disclosure.

FIG. 7 (FIGS. 7-1 and 7-2) is a flow diagram illustrating a process for delivering and/or anchoring an implant to a treatment site in accordance with one or more embodiments of the present disclosure.

FIG. 8 (FIG. 8-1 and 8-2) provides several images associated with the process of FIG. 7 to illustrate aspects of the process according to one or more implementations thereof.

FIGS. 9A and 9B illustrate another example shunt implant in accordance with embodiments of the present disclosure.

FIG. 10 illustrates another example shunt implant in accordance with embodiments of the present disclosure.

FIG. 11 is a flow diagram illustrating a process for delivering and/or anchoring an implant to a treatment site in accordance with one or more embodiments of the present disclosure.

FIG. 12 provides images associated with the process of FIG. 11 to illustrate aspects of the process according to one or more implementations thereof.

### DETAILED DESCRIPTION

The headings provided herein are for convenience only and do not necessarily affect the scope or meaning of the claimed invention.

### Overview

Heart failure is a common and potentially lethal condition affecting humans, with sub-optimal clinical outcomes often resulting in symptoms, morbidity and/or mortality, despite maximal medical treatment. In particular, “diastolic heart failure” refers to the clinical syndrome of heart failure occurring in the context of preserved left ventricular systolic function (ejection fraction) and in the absence of major valvular disease. This condition is characterized by a stiff left ventricle with decreased compliance and impaired relaxation, which leads to increased end-diastolic pressure. Approximately one third of patients with heart failure have diastolic heart failure and there are very few, if any, proven effective treatments.

Symptoms of diastolic heart failure are due, at least in a large part, to an elevation in pressure in the left atrium. Elevated Left Atrial Pressure (LAP) is present in several abnormal heart conditions, including Heart Failure (HF). In addition to diastolic heart failure, a number of other medical conditions, including systolic dysfunction of the left ventricle and valve disease, can lead to elevated pressures in the left atrium. Both Heart Failure with Preserved Ejection Fraction (HFpEF) and Heart Failure with Reduced Ejection Fraction (HFrEF) can exhibit elevated LAP. It has been hypothesized that both subgroups of HF might benefit from a reduction in LAP, which in turn reduces the systolic preload on the left ventricle, Left Ventricular End Diastolic Pressure (LVEDP). It could also relieve pressure on the pulmonary circulation, reducing the risk of pulmonary edema, improving respiration and improving patient comfort.

Pulmonary hypertension (PH) is defined as a rise in mean pressure in the main pulmonary artery. PH may arise from many different causes, but, in all patients, has been shown to increase mortality rate. A deadly form of PH arises in the very small branches of the pulmonary arteries and is known as Pulmonary Arterial Hypertension (PAH). In PAH, the cells inside the small arteries multiply due to injury or disease, decreasing the area inside of the artery and thickening the arterial wall. As a result, these small pulmonary arteries narrow and stiffen, causing blood flow to become restricted and upstream pressures to rise. This increase in pressure in the main pulmonary artery is the common connection between all forms of PH regardless of underlying cause. Despite previous attempts, there is a need for an improved way to reduce elevated pressure in the left atrium, as well as other susceptible heart chambers such as the pulmonary artery.

The present disclosure provides methods and devices (including various implants) for shunting blood within a human body. The term “implant” is used herein according to its plain and ordinary meaning and may refer to any medical implant, frame, valve, shunt, stent, anchor, and/or similar devices for use in treating various conditions in a human body. Implants may be delivered via catheter (i.e., transcatheter) for various medical procedures and may have a generally sturdy and/or flexible structure. The term “catheter” is used herein according to its broad and ordinary meaning and may include any tube, sheath, steerable sheath, steerable catheters, and/or any other type of elongate tubular delivery device comprising an inner lumen configured to slidably receive instrumentation, such as for positioning within an atrium or coronary sinus, including for example delivery catheters and/or cannulas. In some cases, an implant may be composed of a shape-memory alloy (e.g., Nitinol) and/or may have a pre-defined shape and/or structure. The implant may be configured to be shaped and/or compressed to fit into and/or around a catheter. In some cases, an implant may have an elliptical and/or cylindrical form and/or may comprise an interweaving pattern of materials.

Some embodiments described herein provide methods and/or systems for shunting blood within a body of a patient. While some embodiments may be directed to delivery of shunt systems into the coronary sinus ostium (CSO) from the superior vena cava, the methods and/or systems described herein may be applicable to other areas of the body. For example, some methods and/or devices described herein may advantageously be configured for delivery of shunt devices into various challenging cardiovascular anatomies.

Some known and/or conventional shunt devices can include pre-shaped stents and/or similar devices. Such devices can have a generally stiff and/or less-maneuverable structure, particularly when collapsed down during a delivery process. Some embodiments herein advantageously provide flexible and/or maneuverable shunt devices that can facilitate delivery through various pathways in a patient's body.

The following includes a general description of human cardiac anatomy that is relevant to certain inventive features and embodiments disclosed herein and is included to provide context for certain aspects of the present disclosure. In humans and other vertebrate animals, the heart is a hollow muscular organ having four pumping chambers: the left and right atria and the left and right ventricles, each provided with its own one-way valve. The natural heart valves are identified as the aortic, mitral (or bicuspid), tricuspid and pulmonary, and are each mounted in an annulus comprising dense fibrous rings attached either directly or indirectly to the atrial and ventricular muscle fibers. Each annulus defines a flow orifice. The four valves ensure that blood does not flow in the wrong direction during the cardiac cycle; that is, to ensure that the blood does not back flow through the valve. Blood flows from the venous system and right atrium through the tricuspid valve to the right ventricle, then from the right ventricle through the pulmonary valve to the pulmonary artery and the lungs. Oxygenated blood then flows through the mitral valve from the left atrium to the left ventricle, and finally from the left ventricle through the aortic valve to the aorta/arterial system.

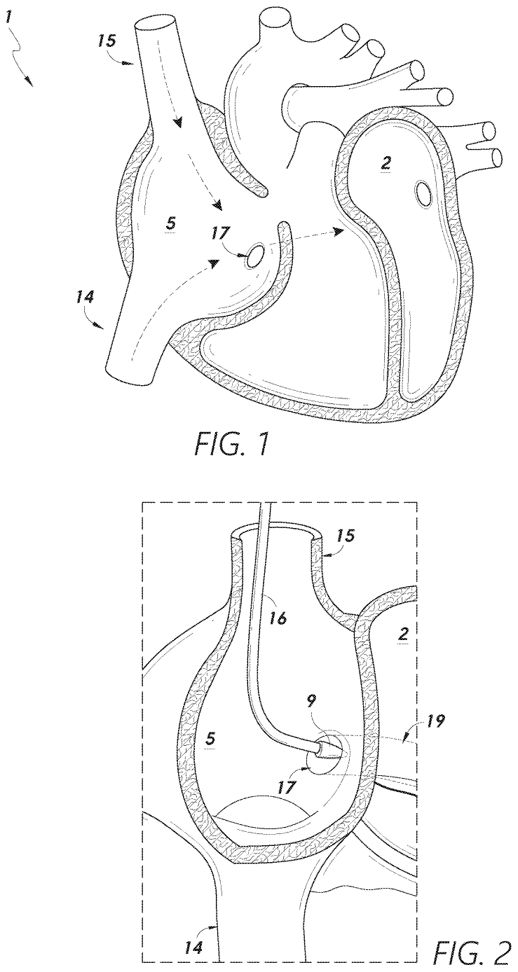

FIG. 1 illustrates several access pathways for maneuvering guidewires and catheters in and around the heart 1 to deploy compressible medical implants (e.g., frames) of the present application. For instance, access may be from above via either the subclavian vein or jugular vein into the superior vena cava (SVC) 15, right atrium (RA) 5 and from there into the coronary sinus ostium (CSO) 17. Alternatively, the access path may start in the femoral vein and through the inferior vena cava (IVC) 14 into the heart 1. Other access routes may also be used, and each typically utilizes a percutaneous incision through which the guidewire and catheter are inserted into the vasculature, normally through a sealed introducer, and from there the physician controls the distal ends of the devices from outside the body.

FIG. 2 depicts an example method for deploying medical implants, wherein a guidewire and/or catheter 16 is/are introduced through the subclavian or jugular vein, through the SVC 15 and into the coronary sinus 19. In some instances, a guidewire may be used to provide a path, after which an introducer sheath (not shown) may be routed along the guidewire and into the patient's vasculature, typically with the use of a dilator 9 and/or atraumatic tip. FIG. 2 shows a deployment catheter 16 extending from the SVC 15 to the CSO 17 and into the coronary sinus 19 of the heart 1. The deployment catheter 16 can be passed through an introducer sheath which can provide a hemostatic valve to prevent blood loss.

The deployment catheter 16 may be about 30 cm long, and the guidewire may be somewhat longer for ease of use. In some embodiments, the deployment catheter may function to form and/or prepare an opening in the wall of the left atrium 2, and a separate placement or delivery catheter may be used for delivery of an expandable implant. In other embodiments, the deployment catheter may be used as both the puncture preparation and implant placement catheter with full functionality. In the present application, the terms “deployment catheter” or “delivery catheter” will be used to represent a catheter or introducer with one or both of these functions.

Since the coronary sinus 19 is largely contiguous around the left atrium 2, there are a variety of possible acceptable placements for implants. The site selected for placement of an implant (e.g., a shunt) may be made in an area where the tissue of the particular patient is less thick or less dense, as determined beforehand by non-invasive diagnostic means, such as a CT scan or radiographic technique, such as fluoroscopy or intravascular coronary echo (IVUS).

Some methods to reduce LAP involve utilizing an implant between the left atrium 2 and the right atrium 5, through the interatrial septum therebetween. This is a convenient approach, as the two structures are adjacent and transseptal access is common practice. However, there may be a possibility of emboli travelling from the right side of the heart to the left, which presents a stroke risk. This event should only happen if the right atrium pressures go above left atrium pressures; primarily during discrete events like coughing, sneezing, Valsalva maneuver, or bowel movements. The anatomical position of the septum would naturally allow emboli to travel freely between the atria if an implant was present and the pressure gradient flipped. This can be mitigated by a valve or filter element in the implant, but there may still be risk that emboli will cross over.

Implanting to the coronary sinus 19 offers some distinct advantages, including that the coronary sinus 19 is much less likely to have emboli present for several reasons. First, the blood draining from the coronary vasculature into the right atrium 5 has just passed through capillaries, so it is essentially filtered blood. Second, the CSO 17 of the coronary sinus 19 in the right atrium 5 is often partially covered by a pseudo-valve called the Thebesian Valve. The Thebesian Valve is not always present, but some studies show it is present in >60% of hearts and it would act as a natural “guard dog” to the coronary sinus to prevent emboli from entering in the event of a spike in right atrium pressure. Third, pressure gradient between the coronary sinus 19 and the right atrium 5 into which it drains is very low, meaning that emboli in the right atrium 5 is likely to remain there. Fourth, in the event that emboli do enter the coronary sinus 19, there will be a much greater gradient between the right atrium 5 and the coronary vasculature than between the right atrium 5 and the left atrium 2. Most likely emboli would travel further down the coronary vasculature until right atrium pressure returned to normal and then the emboli would return directly to the right atrium 5.

Some additional advantages to locating implants between the left atrium 2 and the coronary sinus 19 can include that this anatomy is less mobile and/or more stable than the septum and/or may preserve the septum for later transseptal access for alternate therapies, and it could potentially have other therapeutic benefits. By diverting left atrial blood into the coronary sinus 19, sinus pressures may increase by a small amount. This would cause blood in the coronary vasculature to travel more slowly through the heart, increasing perfusion and oxygen transfer, which would be more efficient and also could help a dying heart muscle to recover. The preservation of transseptal access also is a very significant advantage because HF patients often have a number of other comorbidities like Atrial Fibrillation (AF) and Mitral Regurgitation (MR) and several of the therapies for treating these conditions require a transseptal approach.

An implant may also be positioned within chambers and/or vessels and/or between other cardiac chambers, such as between the pulmonary artery and right atrium 5. Implants may be desirably implanted within the wall of the pulmonary artery using the deployment tools described herein, with the catheters approaching from above and passing through the pulmonary artery. As explained above, pulmonary hypertension (PH) is defined as a rise in mean pressure in the main pulmonary artery. Blood flows through shunt implants from the pulmonary artery into the right atrium 5 if the pressure differential causes flow in that direction, which attenuates pressure and reduces damage to the pulmonary artery. The purpose is to attenuate pressure spikes in the pulmonary artery. Implants may also extend from the pulmonary artery to other heart chambers (e.g., left atrium 2) and/or blood vessels. In some embodiments, implants may further contain a one-way valve for preventing backflow, or a check valve for allowing blood to pass only above a designated pressure.

Tracking shunt delivery systems (e.g., on a guidewire) into the coronary sinus ostium (CSO) 17 from the superior vena cava 15 can often be challenging due at least in part to the CSO-level insertion (CSLI) distance, which may measure a distance between the bottom of the CSO 17 and the bottom of the right atrium 5. If an implant is at least partially stiff and/or creates a stiff section within a portion of the catheter 16, it can make it difficult for surgeons to track the catheter 16 through the CSO 17 and/or other anatomies. In some cases, the catheter 16 can catch on the ridge below the CSO 17 and/or the catheter must form a large “bow” shape into the right atrium 5 (as shown in FIG. 2) when tracking the catheter 16 over a guidewire. In some cases, bowing of the catheter 16 as shown in FIG. 2 can result in a loss of guidewire access due to for example, a guidewire extending from the catheter 16 beyond an access point within the coronary sinus 19.

Some implants described herein may be at least partially compressible and/or expandable. Implants described herein may have various features to simplify and/or improve delivery procedures for surgeons. For example, an implant may be at least partially flexible, compressible, and/or elastic to allow the implant to be shaped and/or molded as necessary/desired to fit into/onto delivery catheters having various sizes and/or shapes.

Moreover, an implant may be configured to fit into various openings created in tissue walls having various sizes and/or shapes. A tissue wall may be situated between a first anatomical chamber (e.g., the coronary sinus 19) and a second anatomical chamber (e.g., the left atrium 2). In some embodiments, an opening may be created through the tissue wall and/or an implant may be configured to fit at least partially within the opening. The opening may represent a blood flow path between the first anatomical chamber and the second anatomical chamber. In some embodiments, an implant may be configured to maintain the opening and/or the blood flow path from the first anatomical chamber to the second anatomical chamber.

### Expandable Shunt Implants

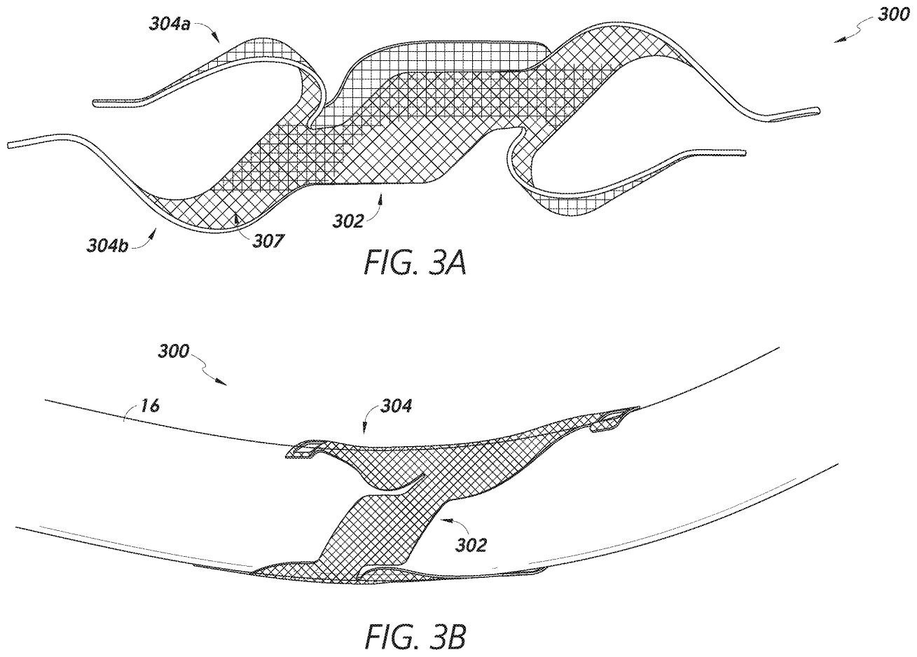

FIGS. 3A and 3B illustrate a flexible shunt implant 300 in accordance with some embodiments. The implant 300 may comprise any of a variety of features and/or components configured to treat various medical conditions. For example, the implant 300 may be configured to maintain an opening in a tissue wall and/or allow blood flow through the tissue wall. In some embodiments, the implant 300 may comprise a central flow portion 302 which may be configured to be situated at least partially within an opening in a tissue wall. The central flow portion 302 may be configured to create and/or maintain an opening between two anatomical chambers. In some embodiments, the implant 300 may comprise multiple separate components which may be attached, connected, and/or otherwise joined to form a single device. For example, the central flow portion 302 may comprise multiple components to form a generally tubular shape which may approximate a shape of an opening in a tissue wall.

The implant 300 may be at least partially composed of braided materials, which may include stainless steel, Nitinol, and/or other metals. At least a portion of the implant 300 may be configured to collapse to a smaller diameter for delivery while maintaining flexibility of the implant 300. In some embodiments, at least a portion of the implant 300 may be configured for delivery into the body on an outer surface and/or outer diameter of a catheter 16, as shown in FIG. 3B. For example, the implant 300 may be configured to be crimped onto the outer surface of the catheter 16. The implant 300 may be crimped down to the catheter as tightly as possible to minimize an increase in diameter of the catheter 16 caused by the implant 300. In some embodiments, at least a portion of the implant 300 may be covered by a tubular sheath (not shown) configured to surround at least a portion of the implant 300. The sheath may be configured to prevent the implant 300 from expanding from a crimped configuration. In some embodiments, additional and/or alternative devices and/or methods may be used to prevent expansion of the implant 300. For example, one or more wires may be attached to the implant 300 to hold the implant 300 tightly against the catheter 16 during delivery into the body.

The implant 300 may be configured to have maximal flexibility during delivery into the body to prevent the implant 300 from hindering bendability of the catheter 16. In some embodiments, the implant 300 may be configured to maintain a level of flexibility while crimped onto the catheter 16. The flexible shunt implant 300 may comprise a central flow portion 302 composed of a network of braided and/or interlocking cords 307, which may include wires, sutures, strings, fibers, and/or various other elongate devices. One or more cords 307 may interact with each other in a weaving/interweaving and/or braiding pattern. For example, a first cord may pass over a second cord, under a third cord, over a fourth cord, and so on. Accordingly, the one or more cords 307 may have at least some flexibility such that a cord 307 may be configured to bend over and/or under other cords 307. For example, one or more cords 307 may be composed of Nitinol and/or another material that may be configured to at least partially bend and/or stretch.

The flow portion 302 may include any number of cords 307. In some embodiments, the flow portion 302 may comprise a single cord 307 configured to interweave with itself. By increasing the number of cords 307 and/or an amount of interweaving of the one or more cords 307, gaps between the cords 307 and/or different sections of a single cord 307 may be minimized to improve prevention and/or reduction of in-growth of tissue. Moreover, each of the cords 307 may have any thickness and may be designed to minimize gaps while maximizing expandability of the flow portion 302.

The flow portion 302 may be configured to be situated at least partially within an opening in a tissue wall. The tissue wall may have a first side and a second side, and the opening may represent a gap through the tissue wall. A “thickness” of the tissue wall may refer to a distance between the first side and a second side of the tissue wall.

The one or more cords 307 of the flow portion 302 may form a cylindrical or other shape to approximate a shape of an opening in a tissue wall. In some embodiments, the opening may be widened in all directions approximately evenly from a puncture point to form an approximately circular opening having a certain diameter. Accordingly, the flow portion 302 may have an at least partially rounded and/or circular form.

In some embodiments, the flexible shunt implant 300 may be in a compacted and/or otherwise flexible form at delivery. For example, at delivery, the one or more cords 307 may be situated relatively close together with minimal gaps between the one or more cords 307. As the tissue wall expands, the one or more cords 307 may gradually separate and/or stretch to create a greater length of the flexible shunt implant 300. In some embodiments, the one or more cords 307 may be configured to stretch in response to expansion of the tissue wall. For example, at delivery, the one or more cords 307 may be in a natural resting state and/or may be only minimally stretched. As the tissue wall expands, at least some of the one or more cords 307 may stretch and/or at least partially separate to create a greater length of the flexible shunt implant 300.

The flexible shunt implant 300 may comprise one or more anchoring arms 304, which may include any means for anchoring a shunt implant to a catheter 16 and/or area of tissue within a body. The one or more anchoring arms 304 (e.g., including a first anchoring arm 304a and/or a second anchoring arm 304b) may be configured to anchor to/into a tissue wall. While the flexible shunt implant 300 is shown having two pairs of anchoring arms 304, the flexible shunt implant 300 may have any number of anchoring arms 304. In some embodiments, the flexible shunt implant 300 may comprise one or more anchoring arms 304 at a first end of the flexible shunt implant 300 and/or one or more anchoring arms 304 at or near a second end of the flexible shunt implant 300. An anchoring arm 304 may attach to and/or extend from any portion of the implant 300, including at one or more cords 307.

In some embodiments, each of the cords 307 and/or anchoring arms 304 may be composed of a common material or different materials. In some embodiments, any of the cords 307 and/or anchoring arms 304 may be composed of Nitinol and/or other metal, plastic, polymer, and/or other material

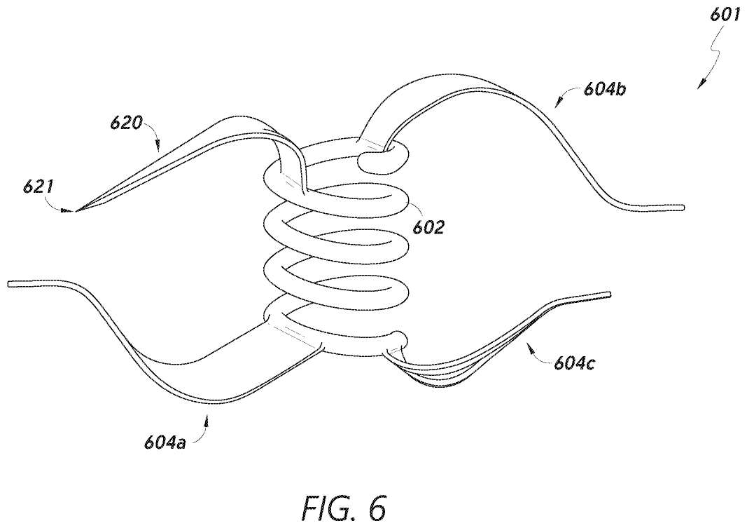

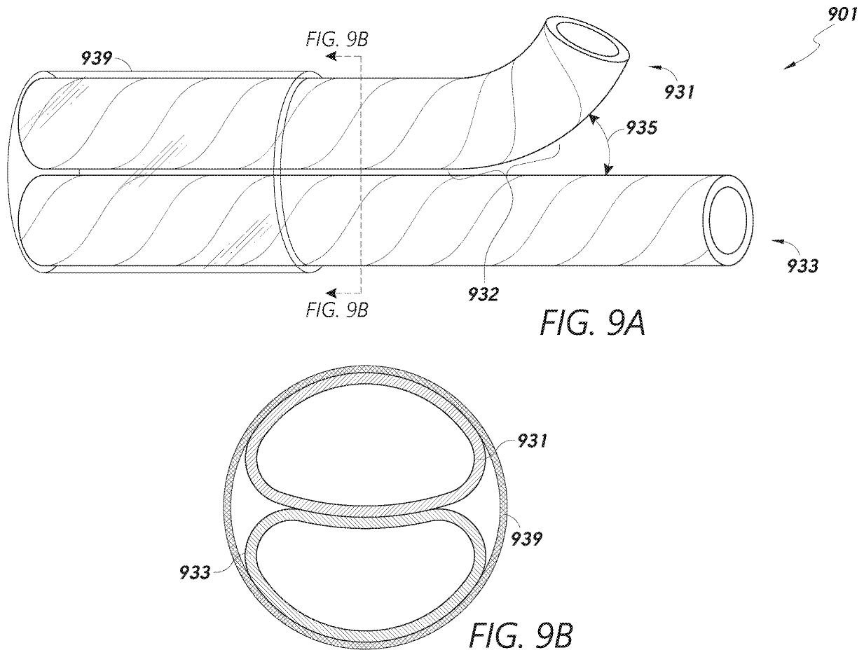

Various features of the shunt implant 300, including the central flow portion 302 and/or anchoring arms 304 described herein, may be applied to the shunt devices described and/or illustrated in other figures of the present application. For example, any description with respect to the shunt implant 300 illustrated in FIGS. 3A and 3B may be similarly applied to the shunt implant 601 in FIG. 6 and/or the shunt implant 901 in FIGS. 9A and/or 9B. Moreover, while other shunts shown and/or described with respect to other figures may not include cords 307 as shown in FIGS. 3A and 3B, it will be understood that the shunts described with respect to other figures may comprise cords 307. Similarly, the various features described with respect to other figures herein may be added to the shunt implant 300 of FIGS. 3A and 3B or other figures herein even if not depicted in or described with respect to each figure. While the shunt implant 300 is shown including both a central flow portion 302 and anchoring arms 304, the shunt implant 300 may in some embodiments not include anchoring arms 304.

In some embodiments, the implant 300 may be configured to be movable between an expanded configuration and a collapsed configuration to facilitate passage through a lumen of a catheter. For example, the central flow portion 302 may be configured to be crimped and/or otherwise compacted to fit around an exterior surface of the catheter 16. Crimping may involve a reduction in diameter of the central flow portion 302 and/or an increase in length of the implant 300. When the implant 300 is crimped, at least a portion of the implant 300 may create resistive force in response to crimping pressure.

The central flow portion 302 may be configured to expand to a pre-defined shape (e.g., the shape and/or size shown in FIG. 3A) and/or size during and/or after removal from a deliver device (e.g., a catheter 16). The implant 300 may further comprise one or more anchoring arms 304, which may include flanges, arms, anchors, and/or other devices. In some embodiments, the one or more anchoring arms 304 (e.g., the first anchoring arm 304a and/or second anchoring arm 304b) may be configured to extend generally perpendicularly (i.e., forming a “T” shape) from the central flow portion. For example, the first anchoring arm 304a and the second anchoring arm 304b may be configured to lay flatly along a common plane. The one or more anchoring arms 304 may have a generally flat, curved, and/or wavy form. In some embodiments, the one or more anchoring arms 304 may be configured to at least partially collapse and/or compress to facilitate passage through the lumen of the catheter and/or may be configured to expand during and/or after delivery within the body to contact and/or attach to a tissue wall. Expansion of the implant 300 may be initiated, for example, by retraction of an outer sheath of the catheter relative to an inner support sheath. The implant 300 may be collapsed (e.g., crimped) into a generally tubular configuration. In some embodiments, the anchoring arms 304 may be configured to spring open when the restraining outer sheath retracts. The anchoring arms 304 may expand generally in opposite directions in a common plane to form a T-shape (see FIG. 3B), as opposed to expanding in a circular fashion. Radiopaque markers on the anchoring arms 304 and/or central flow portion 302 may be provided to facilitate positioning immediately within the body.

A pair of anchoring arms 304 (e.g., a first anchoring arm 304a and a second anchoring arm 304b) may be configured to form a clamping (i.e., pinching) pair of anchoring arms 304. The pairs of anchoring arms 304 may be configured to apply a compressive force to a tissue wall to hold the implant 300 in place. The amount of compressive force may be relatively small to avoid damage to the tissue wall while sufficient to hold the implant 300 in place. For example, gaps separating the pairs of anchoring arms may be calibrated to avoid excessive clamping and/or necrosis of the tissue. The anchoring arms 304 may be configured to secure the implant 300 on generally opposite sides of the tissue wall and/or on generally opposite sides of an opening in the tissue wall. The central flow portion 302 may be configured to be aligned generally perpendicularly to the tissue wall so as to maintain an open flow path between the chambers on either side of the tissue wall.

Components of the implant 300 may be configured to naturally self-expand due to inherent springiness and/or flexibility of the components. For example, various components (e.g., the central flow portion 302 and/or anchoring arms 304) may be at least partially composed of an elastic material such as Nitinol. In some embodiments, the central flow portion 302 may be fabricated by laser cutting a Nitinol tube.

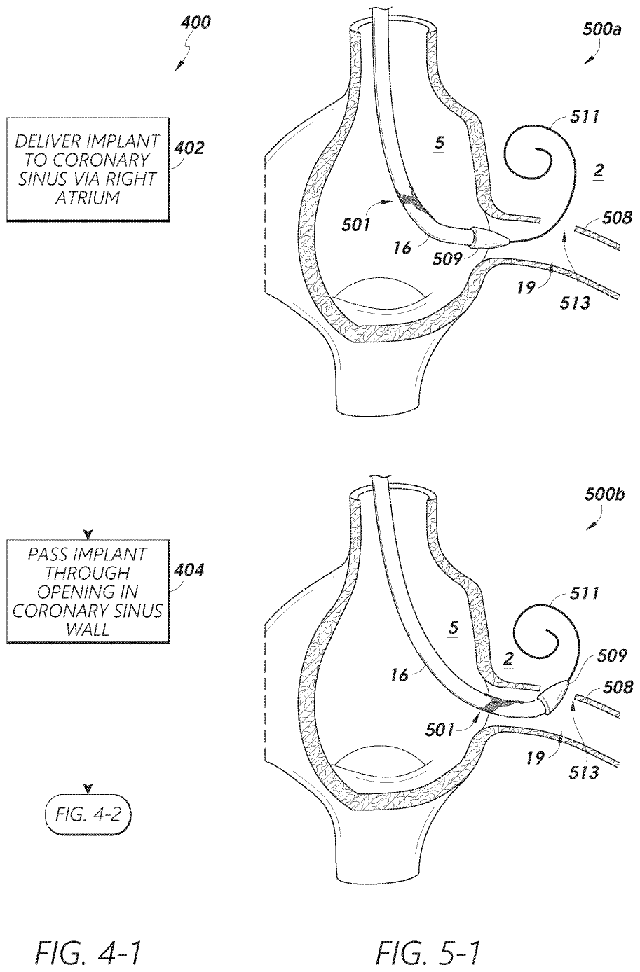

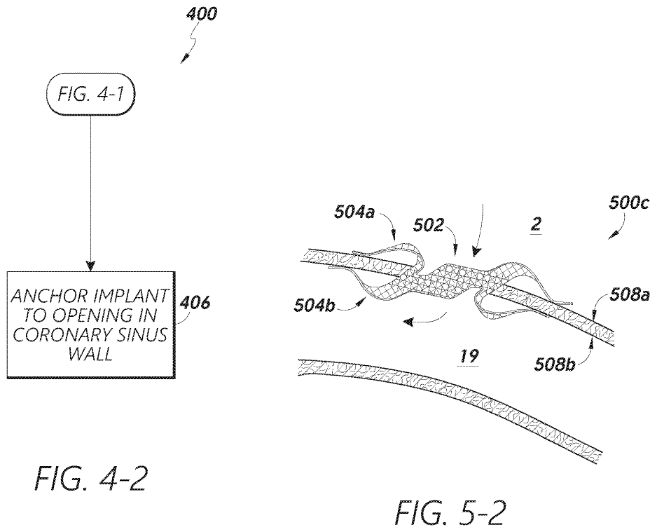

FIG. 4 (FIGS. 4-1 and 4-2) is a flow diagram illustrating a process 400 for delivering and/or anchoring an implant to a treatment site in accordance with one or more embodiments of the present disclosure. FIG. 5 (FIG. 5-1 and 5-2) provides several images associated with the process 400 of FIG. 4 to illustrate aspects of the process according to one or more implementations thereof.

At step 402, the process 400 involves delivering an implant 501 to a pathway within a body, as shown in image 500a of FIG. 5. For example, the implant 501 may be delivered into the coronary sinus 19 via the right atrium 5. The implant 501 may be at least partially compressed onto an outer surface of a catheter 16. In some embodiments, the implant 501 may be at least partially enclosed by a sheath and/or other covering during delivery (see, e.g., the sheath in FIGS. 9A and 9B) to prevent the implant 501 from expanding and/or otherwise impeding delivery to a target location within the body. In some embodiments, wires and/or other mechanisms may be used to hold the implant 501 in a collapsed and/or crimped form. In such embodiments, a sheath may not be needed to hold the implant 501 in the collapsed and/or crimped form.

The implant 501 may have a generally flexible structure such that the catheter 16 may be able to bend as needed to enter the coronary sinus 19 and/or other pathway with minimal restriction from the implant 501. In some embodiments, the catheter 16 may comprise and/or may be attached to an atraumatic tip 509 configured to dilate various pathways and/or otherwise guide the catheter 16 and/or implant 501 to a desired location. Moreover, a guidewire 511 and/or similar device may be delivered to guide the catheter and/or implant 501 to the target location. In some embodiments, an opening 513 in a tissue wall 508 (e.g., a wall of the coronary sinus 19) may be created during the implant 501 delivery process 400 and/or an earlier process. The opening 513 may be created in any of a variety of ways. One example method is the following.

Initially, the guidewire 511 may be advanced, for example, from the right atrium into the coronary sinus 19 through its ostium or opening. A catheter 16 may be advanced over the guidewire. The catheter 16 may be introduced into the body through a proximal end of an introducer sheath. An introducer sheath may provide access to the particular vascular pathway (e.g., jugular or subclavian vein) and may have a hemostatic valve therein. While holding the introducer sheath at a fixed location, the surgeon can manipulate the puncture catheter to the implant site. A puncture sheath having a puncture needle with a sharp tip may be advanced along a catheter 16 and punctured through the wall 508 into, for example, the left atrium 2. A puncture expander may be advanced along the guidewire and through the tissue wall 508 into the left atrium 2. The puncture expander may be, for example, an elongated inflatable balloon. The puncture expander may be inflated radially outward so as to widen the puncture through the tissue wall 508.

At block 404, the process 400 involves passing the implant 501 at least partially through and/or to the opening 513 in the tissue wall 508 (e.g., the coronary sinus 19), as shown in image 500b of FIG. 5. The guidewire 511 may be situated at least partially on a distal side of the tissue wall 508 (e.g., within the left atrium 2) to guide the atraumatic tip 509 and/or implant 501 at least partially to the distal side of the tissue wall 508. Delivering the implant 501 may involve displacing the implant 501 from the catheter 16 when the catheter 16 is delivered to the implant location. For example, when the implant 501 is at least partially situated at or near the opening 513, a sheath covering the implant 501 and/or one or more wires holding the implant 501 in place may be at least partially retracted to allow the implant 501 to expand and/or become displaced from the catheter 16. The implant 501 may be configured to at least partially bend with the catheter 16 into the opening 513.

When the implant 501 is exposed from the catheter 16, the implant 501 may at least partially expand and/or the anchoring arms 504 may at least partially bend. In some embodiments, the implant 501 may be expanded manually and/or may be configured to naturally expand upon being at least partially removed from the catheter 16. However, expansion of the implant 501 may be assisted at least in part. For example, the catheter 16 and/or another surgical tool may be used to press against and/or pull the implant 501 to move the implant 501 towards an expanded shape and/or position.

At block 406, the process 400 involves anchoring the implant 501 within the opening 513 of the tissue wall 508 of the coronary sinus 19 and/or other blood vessel, as shown in image 500c of FIG. 5. The implant 501 may comprise one or more anchoring arms 504 configured to pinch and/or otherwise from an attachment to the tissue wall 508. The implant 501 may be configured to be situated such that blood may flow through the central flow portion 502 of the implant 501 (e.g., from the left atrium 2 into the coronary sinus 19).

The implant 501 may include a central flow portion 502 configured to fit at least partially within the opening 513 in the tissue wall. The central flow portion 502 may be configured to extend along sides of the opening 513 to maintain the opening 513 and/or to prevent ingrowth of tissue. The central flow portion 502 may be configured to allow blood flow through the implant 501. In some embodiments, the anchoring arms 504 may be configured to continue extend from the central flow portion 502 and/or may be configured to maintain the opening 513. The central flow portion 502 and/or anchoring arms 504 may comprise a network and/or pattern of struts and/or cords configured to form any suitable pattern and/or shape allowing blood flow through the central flow portion 502 and/or anchoring arms 504.

In some embodiments, a first anchoring arm 504a may be configured to contact and/or attach to a first side 508a of the tissue wall 508 and/or a second anchoring arm 504b may be configured to contact and/or attach to a second side 508b of the tissue wall 508. The first anchoring arm 504a and the second anchoring arm 504b may be configured to establish a pincer grip on the tissue wall 508 by pressing simultaneously against the first side 508a of the tissue wall 508 and the second side 508b of the tissue wall 508, respectively.

The implant 501 may comprise two sets of anchoring arms 504, each configured to anchor to different portions of the tissue wall 508. For example, a first set of anchoring arms 504 (including the first anchoring arm 504a and the second anchoring arm 504b) may be configured to establish a pincer grasp on a first portion of the tissue wall 508 while a second set of anchoring arms may be configured to establish a pincer grasp on a second portion of the tissue wall 508.

In some embodiments, one or more anchoring arms 504 may be configured to penetrate and/or hook onto the tissue wall 508. For example, one or more anchoring arms 504 may comprise one or more hooks extending from the anchoring arms 504 configured to penetrate the tissue wall 508 to establish a more secure attachment to the tissue wall 508.

The one or more anchoring arms 504 may be configured to naturally bend towards the tissue wall 508 upon delivery at the opening 513 in the tissue wall 508. For example, when the implant 501 is removed from a delivery device (e.g., a catheter), the implant 501 may naturally assume the form shown in image 500c. In some embodiments, the implant 501 may be shape-set and/or may be at least partially composed of a shape-memory alloy (e.g., Nitinol). For example, the implant 501 may be shape-set such that the first anchoring arm 504a and the second anchoring arm 504b may be configured to be pressed together and/or near each other such that when the tissue wall 508 is situated between the first anchoring arm 504a and the second anchoring arm 504b, the first anchoring arm 504a and the second anchoring arm 504b may be configured to securely pinch the tissue wall 508.

FIG. 6 illustrates another example shunt implant 601 in accordance with embodiments of the present disclosure. In some embodiments, the implant 601 may comprise a central flow portion 602 having a coiled and/or braided structure. For example, the central flow portion 602 may comprise one or more coiled cords, wires, and/or other elongate devices. The one or more cords may be formed in coils to form an inner lumen to allow blood flow through the central flow portion 602. While the implant 601 is shown in FIG. 6 having a coiled central flow portion 602, the implant 601 may additionally or alternatively comprise braided portions, similarly to the implant 300 of FIGS. 3A and 3B. Moreover, the implant 300 of FIGS. 3A and 3B may additionally or alternatively comprise coiled portions, similarly to the implant 601 of FIG. 6.

In some embodiments, the implant 601 may comprise a puncture element 620 configured to puncture through a tissue wall and/or to anchor to a tissue wall. The puncture element 620 may advantageously allow for surgeons to deliver the implant 601 without having to deliver a separate puncture element (e.g., a needle) prior to delivering the implant 601. In other words, creating an opening in a tissue wall and delivering the implant 601 to the opening in the tissue wall may be performed as a single-step process without having to remove devices from the body prior to delivering the implant 601.

As shown in FIG. 6, the puncture element 620 may be configured to extend from the central flow portion 602. In some embodiments, the puncture element 620 and/or one or more anchoring arms 604 may be configured to extend from the central flow portion 602. For example, the puncture element 620 may replace an anchoring arm 604 and/or may be used in conjunction with one or more anchoring arms 604. The puncture element 620 may be configured to perform functions similarly to an anchoring arm 604. For example, the puncture element 620 may be configured to establish a pincer grasp on a tissue wall together with a first anchoring arm 604a. A second anchoring arm 604b and/or third anchoring arm 604c may be configured to establish a pincer grasp on a different portion of a tissue wall.

The puncture element 620 may have any of a variety of forms and/or structures. In some embodiments, the puncture element 620 may have a generally conical form with a pointed tip 621 at one end and increasing in diameter and/or width from the pointed tip 621 to the central flow portion 602. The puncture element 620 may additionally or alternatively have a generally cylindrical structure (e.g., similarly to a needle) and forming the pointed tip 621 at an end portion. In another example, the puncture element 620 may have a general flat structure and/or may increase in width from the pointed tip 621 to the central flow portion 602. The puncture element 620 may be configured to form an opening in a tissue wall having a suitable size to fit the crimped/compressed implant 601 and/or a catheter through the opening. Accordingly, the puncture element 620 may be configured to at least partially increase in width and/or diameter from the pointed tip to increase the size of the opening created by the puncture element 620. Following delivery through an opening in a tissue wall, the puncture element 620 may be configured to contact and/or at least partially penetrate a surface of a tissue wall to anchor the implant 601 at the opening. For example, the pointed tip 621 may be configured to establish a single point of contact at the tissue wall. By establishing a single point of contact at the tissue wall, the puncture element 620 may be configured to assist in anchoring the implant 601 to the tissue wall while also minimizing surface contact to minimize in-growth of tissue around the puncture element and/or at the central flow portion 602.

The implant 601 may have a generally flexible structure. While the implant 601 is shown having a coiled structure, the implant 601 may additionally or alternatively comprise braided and/or interweaving elements to provide a measure of flexibility to the implant 601. Moreover, the puncture element 620 may be composed of an at least partially flexible material (e.g., Nitinol and/or thin portions of stainless steel) to allow the puncture element 620 to at least partially bend during delivery to the target location.

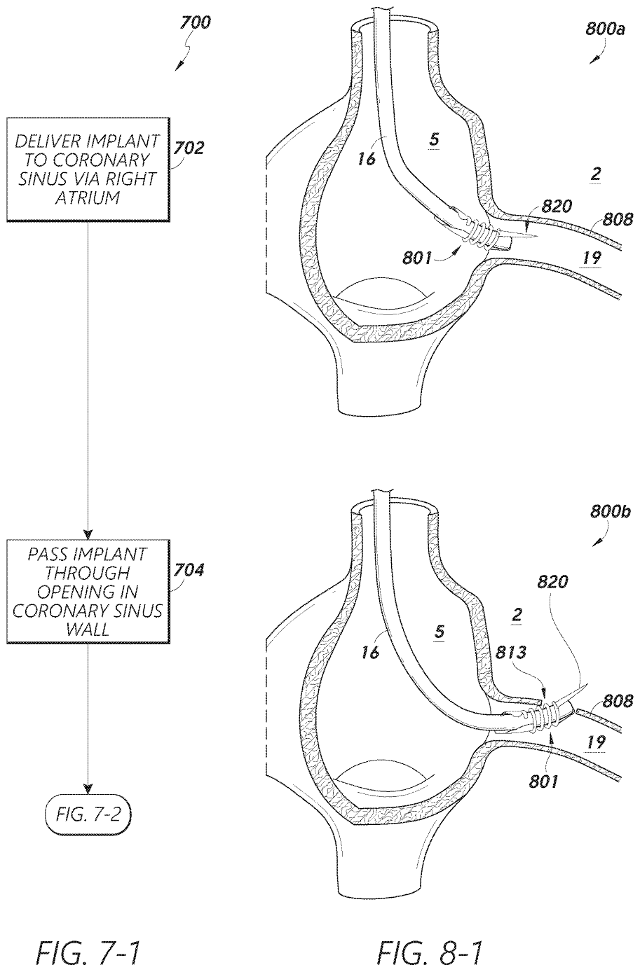

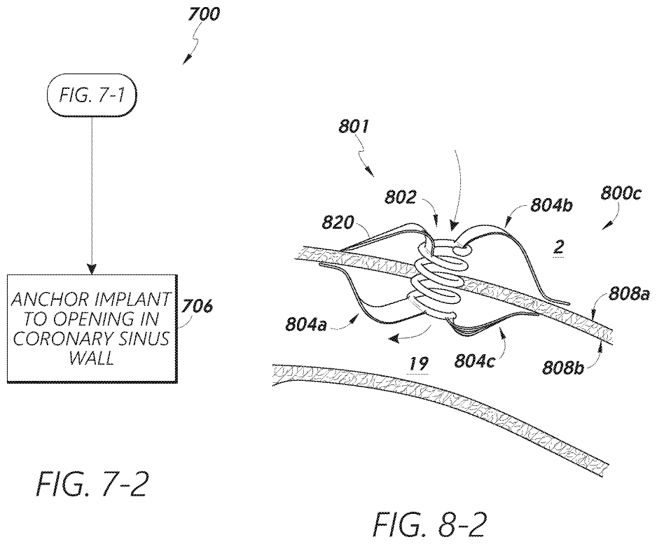

FIG. 7 (FIGS. 7-1 and 7-2) is a flow diagram illustrating a process 700 for delivering and/or anchoring an implant to a treatment site in accordance with one or more embodiments of the present disclosure. FIG. 8 (FIG. 8-1 and 8-2) provides several images associated with the process 700 of FIG. 7 to illustrate aspects of the process 700 according to one or more implementations thereof.

At step 702, the process 700 involves delivering an implant 801 to one or more flow pathways within a body, as shown in image 800a of FIG. 8. For example, the implant 801 may be delivered into the coronary sinus 19 via the right atrium 5. The implant 801 may be at least partially compressed onto an outer surface of a catheter 16. While the implant 801 is shown in FIG. 8 having a coiled structure, the implant 801 may have any suitable structure, which may include one or more at least partially braided portions. In some embodiments, the implant 801 may be configured to be at least partially enclosed by a sheath and/or other covering during delivery (see, e.g., the sheath in FIGS. 9A and 9B) to prevent the implant 801 from expanding and/or otherwise impeding delivery to a target location within the body. In some embodiments, wires and/or other mechanisms may be used to hold the implant 801 in a collapsed and/or crimped form.

The implant 801 may have a generally flexible structure such that the catheter 16 may be able to bend as needed to enter the coronary sinus 19 and/or other pathway with minimal restriction from the implant 801. In some embodiments, the implant 801 may comprise at least one puncture element 820 configured to puncture and/or anchor to a tissue wall. The puncture element 820 may extend from a central portion and/or anchoring arm of the implant 801. In some embodiments, the puncture element 820 may be used in place of an anchoring arm and/or may be in combination with other anchoring arms. The puncture element 820 may be composed of any suitable material and/or combination of materials, which may include Nitinol and/or other metals. In some embodiments, the puncture element 820 may be configured to at least partially bend to allow the puncture element 820 and/or implant 801 to be more easily navigated to a target location (e.g., a wall of the coronary sinus 19).

In some embodiments, the puncture element 820 may have a generally tapered structure and/or may be configured to dilate various pathways and/or otherwise guide the catheter 16 and/or implant 801 to a desired location. The puncture element 820 may have a generally conical shape such that the puncture element 820 may be configured to create a circular-shaped opening 813 in the tissue wall 808. Moreover, a guidewire and/or similar device may be delivered to guide the catheter and/or implant 801 to the target location, as shown in image 800b of FIG. 8.

At block 704, the process 700 involves passing the implant 801 at least partially through and/or to the opening 813 in the tissue wall 808 (e.g., the coronary sinus 19) created by the puncture element 820, as shown in image 800b of FIG. 8. The puncture element 820 may be configured to pressed through the tissue wall to create the opening 813. In some embodiments, the puncture element 820 may be configured to create an opening 813 having a suitable size and/or width to allow the catheter 16 and/or implant 801 body to fit at least partially through the opening 813. The guidewire may be situated at least partially on a distal side of the tissue wall 808 (e.g., within the left atrium 2) to guide the catheter 16 and/or implant 801 at least partially to the distal side of the tissue wall 808. Delivering the implant 801 may involve displacing the implant 801 from the catheter 16 when the catheter 16 is delivered to the implant location. For example, when the implant 801 is at least partially situated at or near the opening 813, a sheath covering the implant 801 and/or one or more wires holding the implant 801 in place may be at least partially retracted to allow the implant 801 to expand and/or become displaced from the catheter 16. The implant 801 may be configured to at least partially bend with the catheter 16 into the opening 813.

When the implant 801 is removed from the catheter 16, the implant 801 may be configured to at least partially expand and/or the anchoring arms 804 may at least partially bend. In some embodiments, the implant 801 may be expanded manually and/or may be configured to naturally expand upon being at least partially removed from the catheter 16. However, expansion of the implant 801 may be assisted at least in part. For example, the catheter 16 and/or another surgical tool may be used to press against and/or pull the implant 801 to move the implant 801 towards an expanded shape and/or position.

At block 706, the process 700 involves anchoring the implant 801 within the opening 813 of the tissue wall 808, as shown in image 800c of FIG. 8. The implant 801 may comprise one or more anchoring arms 804 configured to pinch and/or otherwise from an attachment to the tissue wall 808. The implant 801 may be configured to be situated such that blood may flow through the central flow portion 802 of the implant 801 (e.g., from the left atrium 2 into the coronary sinus 19).

The implant 801 may include a central flow portion 802 configured to fit at least partially within the opening 813 in the tissue wall. The central flow portion 802 may be configured to extend along sides of the opening 813 to maintain the opening 813 and/or to prevent ingrowth of tissue. The central flow portion 802 may be configured to allow blood flow through the implant 801. In some embodiments, the anchoring arms 804 may be configured to continue extend from the central flow portion 802 and/or may be configured to maintain the opening 813.

In some embodiments, a first anchoring arm 804a may be configured to contact and/or attach to a second side 808b of the tissue wall 808 and/or the puncture element 820 may be configured to contact and/or attach to a first side 808a of the tissue wall 808. The first anchoring arm 804a and the puncture element 820 may be configured to establish a pincer grip on the tissue wall 808 by pressing simultaneously against the first side 808a of the tissue wall 808 and the second side 808b of the tissue wall 808, respectively. The implant 801 may comprise a set of anchoring arms (e.g., a second anchoring arm 804b and/or a third anchoring arm 804c) opposite the first anchoring arm 804a and the puncture element 820, each configured to anchor to different portions of the tissue wall 808.

In some embodiments, one or more anchoring arms 804 may be configured to penetrate and/or hook onto the tissue wall 808. For example, one or more anchoring arms 804 may comprise one or more hooks extending from the anchoring arms 804 configured to penetrate the tissue wall 808 to establish a more secure attachment to the tissue wall 808.

The one or more anchoring arms 804 may be configured to naturally bend towards the tissue wall 808 upon delivery at the opening 813 in the tissue wall 808. For example, when the implant 801 is removed from a delivery device (e.g., a catheter), the implant 801 may naturally assume the form shown in image 800c. In some embodiments, the implant 801 may be shape-set and/or may be at least partially composed of a shape-memory alloy (e.g., Nitinol). For example, the implant 801 may be shape-set such that the first anchoring arm 804a and the puncture element 820 may be configured to be pressed together and/or near each other such that when the tissue wall 808 is situated between the first anchoring arm 804a and the puncture element 820, the first anchoring arm 804a and the puncture element 820 may be configured to securely pinch the tissue wall 808.

In some embodiments, the puncture element 820 may be configured to establish a single point of contact with the tissue wall 808. For example, a pointed tip of the puncture element 820 may be configured to penetrate the tissue wall. In this way, the puncture element 820 may advantageously limit tissue growth on the puncture element 820 and/or at the implant 801 due to a relatively low amount of surface contact between the puncture element 820 and the tissue wall 808.

FIGS. 9A and 9B illustrate another example shunt implant 901 in accordance with embodiments of the present disclosure. In some embodiments, the implant 901 may comprise a bi-lateral structure comprising two or more portions including a first portion 931 (e.g., first shunt) and a second portion 933 (e.g., second shunt). The first portion 931 and/or the second portion 933 may comprise generally tubular devices having inner lumens configured to receive and/or allow blood flow. In some embodiments, the first portion 931 and/or the second portion 933 may be at least partially fluid-tight and/or may be configured to allow blood flow through the tubular walls of the portions. For example, the first portion 931 and/or the second portion 933 may be formed at least partially of a braided and/or coiled network of cords and/or other devices providing gaps to allow blood flow.

The first portion 931 may be configured to allow shunting of blood flow from a first chamber (e.g., the left atrium) into a second chamber and/or blood pathway (e.g., the coronary sinus). Moreover, the first portion 931 may advantageously be configured to maintain an opening in a tissue wall. The second portion 933 may be configured to allow normal blood flow through a blood pathway (e.g., the coronary sinus). Moreover, the second portion 933 may be configured to at least partially prevent blood flow through the flow pathway from entering the first chamber. The first portion 931 and/or second portion 933 may have a generally tubular shape to allow the first portion 931 and/or second portion 933 to be able to bend and/or otherwise maintain a relatively high level of flexibility to simplify delivery of the implant 901 into the body.

In some embodiments, at least a portion of the first portion 931 and/or the second portion 933 may be at least partially fluid tight and/or may comprise one or more areas (e.g., a first area 932) at least partially composed of a material or materials configured to prevent blood flow through the one or more areas. For example, the first area 932 and/or other areas of the first portion 931 and/or the second portion 933 may be at least partially composed of a generally solid material that may not form gaps to thereby prevent blood flow through the first area 932. The first area 932 and/or other areas may be configured to be situated at least partially below an opening of a tissue wall and/or may be configured to redirect blood flow through the opening. For example, blood may flow generally perpendicularly through an opening due to blood pressure characteristics of the chambers on either side of the opening. Such blood flow may have a relatively high velocity and/or may be potentially damaging to various tissue walls, particularly if the blood flows directly into a tissue wall. The first portion 931 and/or the second portion 933 may advantageously be configured to prevent at least a portion of blood flow from flowing directly into a tissue wall by redirecting the blood along a flow pathway (e.g., along the coronary sinus) rather than directly into a tissue wall. For example, blood flow may be deflected and/or redirected by the first area 932 of the first portion 931.

The first portion 931 and/or the second portion 933 may be configured to be at least partially enclosed by a sheath 939 which may be configured to hold the first portion 931 and/or the second portion 933 together. The sheath may be at least partially composed of cloth and/or other material(s). FIG. 9B provides a cross-sectional view illustrating the first portion 931 and/or the second portion 933 within the sheath 939. In some embodiments, the sheath 939 may be configured to be extended entirely over the first portion 931 and/or the second portion 933. When the implant 901 is delivered to target location within a body, the sheath 939 may be configured to be at least partially retracted to expose at least a portion of the first portion 931 and/or the second portion 933. When the first portion 931 is exposed and/or removed from the sheath 939, the first portion 931 may be configured to at least partially bend to form an angle of separation 935 between the first portion 931 and the second portion 933. The angle of separation 935 may be any value between 0° and 90°.

In some embodiments, at least part of the first portion 931 may be configured to naturally bend away from the second portion 933. For example, the first portion 931 may be at least partially composed of a shape memory alloy (e.g., Nitinol) and/or may be shape-set to assume the bent form shown in FIG. 9A when removed from the sheath 939.

One or more anchoring elements may be attached to the first portion 931 and/or the second portion 933 to anchor the first portion 931 and/or the second portion 933 to a tissue wall. For example, one or more anchoring elements may extend from an end portion of the first portion 931 to allow the first portion 931 to anchor to a tissue wall after passing through an opening in the tissue wall.

The second portion 933 may be configured for retention of the first portion 931 within an opening of a tissue wall. For example, the second portion 933 may be configured to remain within a blood flow pathway while the first portion 931 extends away from the second portion 933 (e.g., generally perpendicularly from the second portion 933) to enter the opening. The second portion 933 may be sized and/or positioned such that the second portion 933 may be configured to press against walls of the blood flow pathway to prevent the first portion 931 from dropping and/or receding out of the opening after the first portion 931 has entered the opening.

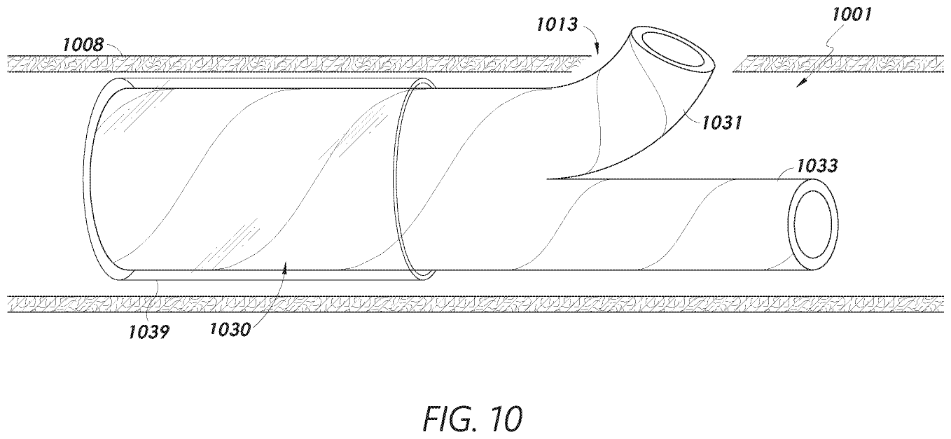

FIG. 10 illustrates another example shunt implant 1001 in accordance with embodiments of the present disclosure. The implant 1001 is shown in FIG. 10 within a blood flow pathway between tissue walls 1008. In some embodiments, an implant 1001 may comprise a first portion 1031 and/or a second portion 1033 configured to extend from a common base portion 1030.

The first portion 1031 and/or the second portion 1033 may comprise generally tubular devices having inner lumens configured to allow blood flow. In some embodiments, the first portion 1031 and/or the second portion 1033 may be at least partially fluid-tight and/or may be configured to allow blood flow through the tubular walls of the portions. For example, the first portion 1031 and/or the second portion 1033 may be formed at least partially of a braided and/or coiled network of cords and/or other devices providing gaps to allow blood flow. In some embodiments, at least a portion of the first portion 1031 and/or the second portion 1033 may be at least partially fluid tight and/or may comprise a one or more areas at least partially composed of a material or materials configured to prevent blood flow through the one or more areas (see, e.g., the first area 932 of FIGS. 9A and 9B).

The first portion 1031 and/or the second portion 1033 may be configured to be at least partially enclosed by a sheath 1039 which may be configured to hold the first portion 1031 and/or the second portion 1033 together. In some embodiments, the sheath 1039 may be configured to be extended entirely over the first portion 1031 and/or the second portion 1033. When the implant 1001 is delivered to target location within a body, the sheath 1039 may be configured to be at least partially retracted to expose at least a portion of the first portion 1031 and/or the second portion 1033. When the first portion 1031 is exposed and/or removed from the sheath 1039, the first portion 1031 may be configured to at least partially bend to form an angle of separation between the first portion 1031 and the second portion 1033. The angle of separation may be any value between 0° and 90°.

In some embodiments, the first portion 1031 may be configured to extend through an opening 1013 created in a tissue wall 1008. The opening 1013 may be formed prior to delivery of the implant 1001 as described previously herein. In some embodiments, the second portion 1033 and/or base portion 1030 may be sized and/or positioned to retain the first portion 1031 within the opening 1013 and/or to prevent the first portion 1031 from receding and/or dropping back completely into the blood flow pathway in which the second portion 1033 may be situated.

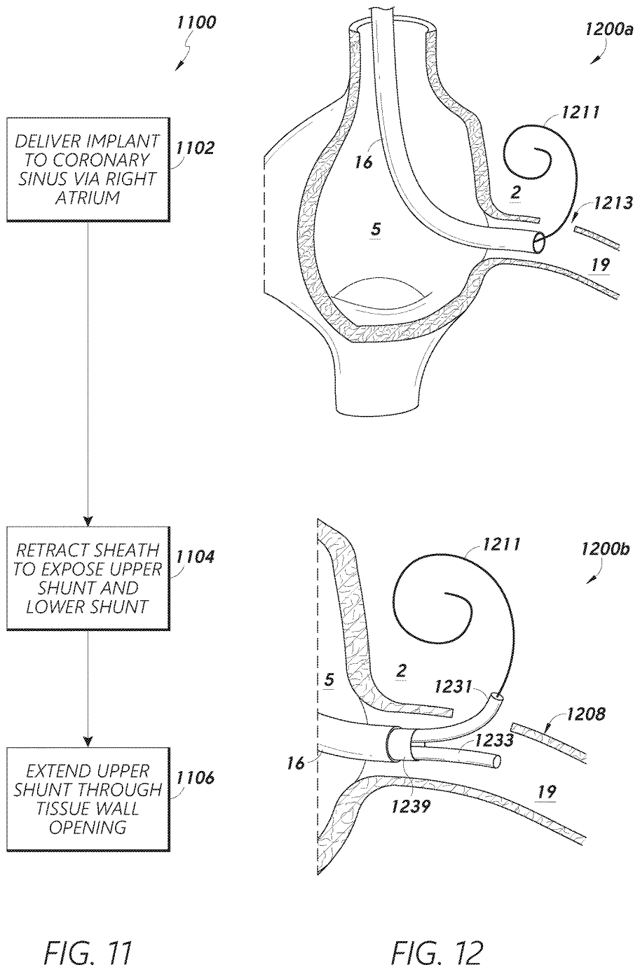

FIG. 11 is a flow diagram illustrating a process 1100 for delivering and/or anchoring an implant to a treatment site in accordance with one or more embodiments of the present disclosure. FIG. 12 provides several images associated with the process 1100 of FIG. 11 to illustrate aspects of the process 1100 according to one or more implementations thereof.

At step 1102, the process 1100 involves delivering an implant to one or more pathways within a body, as shown in image 1200a of FIG. 12. For example, the implant may be delivered into the coronary sinus 19 via the right atrium 5. The implant may be configured to be delivered via a catheter 16. In some embodiments, the implant may be configured to be at least partially enclosed by a sheath 1239 and/or other covering during delivery (see image 1200b of FIG. 12) to prevent the implant from expanding and/or otherwise impeding delivery to a target location within the body. In some embodiments, wires and/or other mechanisms may be used to hold the implant in a collapsed and/or crimped form. The implant may comprise one or more tubular portions (e.g., shunts) having lumens configured to receive blood flow and/or facilitate blood flow through and/or into the pathways. The implant may be delivered near (e.g., below) an opening 1213 of a tissue wall 1208. The opening 1213 may have been created during an earlier stage of a delivery process and/or a guidewire 1211 may have been delivered through the opening 1213 to guide the catheter 16.

At step 1104, the process 1100 involves retracting the sheath 1239 and/or catheter 16 to expose at least a portion of first (e.g., upper) shunt 1231 and/or second (e.g., lower) shunt 1233 and/or to allow the first shunt 1231 to at least partially separate from the second shunt 1233, as shown in image 1200b of FIG. 12. The sheath 1239 and/or catheter 16 may be configured to prevent expansion and/or separation of the first shunt 1231 and/or second shunt 1233. When the first shunt 1231 and/or second shunt 1233 are exposed and/or at least partially removed from the sheath 1239 and/or catheter 16, the first shunt 1231 and/or second shunt 1233 may be configured to assume a natural pre-determined form. For example, the first shunt 1231 and/or second shunt 1233 may be at least partially composed of one or more shape-memory materials (e.g., Nitinol) to cause the first shunt 1231 and/or second shunt 1233 to naturally gravitate towards a pre-determined shape. In some embodiments, at least a portion of the first shunt 1231 may be configured to naturally bend away from the second shunt 1233 when the first shunt 1231 is exposed.

At step 1106, the process 1100 involves extending the first shunt 1231 through the opening 1213 in the tissue wall 1208. For example, the first shunt 1231 may be configured to bend away from the second shunt 1233 to create some separation between the first shunt 1231 and the second shunt 1233. The first shunt 1231 may be configured to extend through an opening 1213 created in the tissue wall 1208. In some embodiments, the first shunt 1231 and/or the catheter 16 may be configured to track along the guidewire 1211 delivered previously into the body.

The second shunt 1233 may be configured to remain within the blood flow pathway (e.g., the coronary sinus 19) while the first shunt 1231 extends out of the blood flow pathway and/or into a chamber (e.g., the left atrium 2) on a distal side of the tissue wall 1208. In some embodiments, the second shunt 1233 may be configured to at least partially stabilize the first shunt 1231 and/or to prevent the first shunt 1231 from receding out of the opening 1213 in the tissue wall 1208. For example, the second shunt 1233 may be configured to press against one or more walls of the blood flow pathway to prevent the first shunt 1231 from dropping out of the opening 1213.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

1. (canceled)

2. An expandable shunt configured to be self-expandable from a collapsed state to an expanded state, the shunt comprising:
a central flow portion configured to be implanted within an opening in a tissue wall, the central flow opening having a first end, a second end, and a lumen extending from the first end to the second end;
a first anchoring arm extending from the first end of the central flow portion and configured to contact a first side of the tissue wall; and
a second anchoring arm extending from the second end of the central flow portion and configured to contact a second side of the tissue wall;
wherein the central flow portion, the first anchoring arm, and the second anchoring arm comprise a braided or woven structure.

3. The expandable shunt of claim 2, wherein the braided or woven structure is formed from a plurality of interlaced cords configured to reduce ingrowth of tissue on the central flow portion of the shunt.

4. The expandable shunt of claim 3, wherein the cords comprise wires.

5. The expandable shunt of claim 3, wherein the cords comprise fibers.

6. The expandable shunt of claim 3, wherein the cords are made of Nitinol.

7. The expandable shunt of claim 3, wherein the cords are configured to stretch when a length of the shunt increases.

8. The expandable shunt of claim 3, wherein the braided or woven structure comprises openings between adjacent cords.

9. The expandable shunt of claim 2, wherein the first anchoring arm and the second anchoring arm extend from axially opposed locations on the first end and the second end, respectively, of the central flow portion.

10. The expandable shunt of claim 9, wherein:
the first anchoring arm extends from the first end of the central flow portion in a direction away from the second anchoring arm and then curves back toward the second anchoring arm, the first anchoring arm having a free end portion spaced laterally from the central flow portion;
the second anchoring arm extends from the second end of the central flow portion in a direction away from the first anchoring arm and then curves back toward the first anchoring arm, the second anchoring arm having a free end portion spaced laterally from the central flow portion; and
the free end portions of the first and second anchoring arms are configured to establish a pincher grasp on the tissue wall outside of the opening in the tissue wall.

11. The expandable shunt of claim 10, wherein:
the first anchoring arm comprises two first anchoring arms extending from diametrically opposed locations on the first end of the central flow portion;
the second anchoring arm comprises two second anchoring arms extending from diametrically opposed locations on the second end of the central flow portion; and
one of first anchoring arms and one of the second anchoring arms form a first pair of anchoring arms that is configured to establish a pincher grasp on the tissue wall;
another one of the first anchoring arms and another one of the second anchoring arms form a second pair of anchoring arms that is configured to establish a pincher grasp on the tissue wall.

12. The expandable shunt of claim 2, wherein the central flow portion is a cylindrical section of the braided or woven structure.

13. An expandable shunt configured to be self-expandable from a collapsed state to an expanded state, the shunt comprising:
a plurality of interlaced cords forming a central flow portion configured to be implanted within an opening in a tissue wall, the central flow opening having a first end, a second end, and a lumen extending from the first end to the second end, wherein the plurality of interlaced cords is configured to reduce tissue ingrowth on the central flow portion; and
at least one pair of first and second anchoring arms extending from axially opposed locations on the first end and the second end, respectively, of the central flow portion, wherein the first and second anchoring arms are configured to establish a pincher grasp on the tissue wall outside of the opening in the tissue wall.

14. The expandable shunt of claim 13, wherein the cords form a braided structure forming the central flow portion.

15. The expandable shunt of claim 13, wherein the cords form a woven structure forming the central flow portion.

16. The expandable shunt of claim 13, wherein the cords comprise wires.

17. The expandable shunt of claim 13, wherein the plurality of interlaced cords forms the at least one pair of first and second anchoring arms.

18. An expandable shunt configured to be self-expandable from a collapsed state to an expanded state, the shunt comprising:
a central flow portion configured to be implanted within an opening in a tissue wall, the central flow opening having a first end, a second end, and a lumen extending from the first end to the second end, wherein the central flow portion comprises a woven structure;
a plurality of first anchoring arms extending from the first end of the central flow portion and configured to contact a first side of the tissue wall; and
a plurality of second anchoring arms extending from the second end of the central flow portion and configured to contact a second side of the tissue wall;
wherein the plurality of first anchoring arms and the plurality of second anchoring arms are configured to establish a pincher grasp at discrete, spaced apart locations on the tissue wall outside of the opening in the tissue wall.

19. The expandable shunt of claim 18, wherein the woven structure is formed from a plurality of interwoven cords configured to reduce tissue ingrowth on the central flow portion.

20. The expandable shunt of claim 19, wherein the cords comprise fibers.

21. The expandable shunt of claim 19, wherein the cords comprise wires.
